(19) United States
(12) Patent Application Publication
(10) Pub. No.: US 2014/0336540 A1
(43) Pub. Date: Nov. 13, 2014

(54) CLEANING AND MASSAGING SYSTEM

(71) Applicant: Shawn Chen, Kansas City, MO (US)

(72) Inventor: Shawn Chen, Kansas City, MO (US)

(21) Appl. No.: 13/888,813

(22) Filed: May 7, 2013

(51) Int. Cl. A61H 23/06; A61N 7/00

(52) U.S. Cl. CPC A61H 23/06; A61N 7/00; A61N 2007/0034
USPC 601/2; 601/46

(57) ABSTRACT

A cleaning and massaging system includes a water resistant housing having a top surface and a bottom surface; a display and one or more buttons coupled to the top portion; a processor inside the housing; and a vibration generator driven by the processor to vibrate on user command.

### BACKGROUND

The present invention generally relates to the field of massage devices.

In the past there have been two general types of commercially available massagers which could be used by persons to assist them in giving themselves or others a muscular and skin massage. In one type, a vibrating massager is carried on the back of a person's hand and the hand, with vibratory motion imparted to it by the massager, is utilized to perform the actual massaging of the body. In the other type, the massager is placed directly against the portion of the body to be massaged and may or may not be held by the person during usage.

With respect to the latter type of massagers, generally the head portion of the massager, i.e., the portion which includes the body-contacting surface, is securely attached or is an integral part of the rest of the massager housing so that there is no relative movement between the body-contacting surface and the rest of the massager. Also, these prior body-contacting type massagers usually used electro-magnetic means to impart vibratory motion to the body-contacting surface.

### SUMMARY

A cleaning and massaging system includes a water resistant housing having a top surface and a bottom surface; a display and one or more buttons coupled to the top portion; a processor inside the housing; and a vibration generator driven by the processor to vibrate on user command.

Implementation of the above system can include one or more of the following. The vibration generator can be a motor. User-replaceable head can be used. The user-replaceable brush heads can be selected from a group consisting of: Body Brush Head, Normal Skin Brush Head, Sensitive Skin Brush Head, Toning Applicator, and Hydration/Exfoliation Applicator. A motor driven blade can be used generate vibration. The motor driven blade can be eccentric. One of the buttons can be an intensity button to select five different intensity levels. The intensity levels include intensity 1 for sensitive skin, intensity 2 for normal skin, intensity 3 for deep pore skin, and intensity 4 and 5 for body skin treatment. One of the buttons can be a mode button to select three different modes.

Advantages of the preferred embodiments may include one or more of the following. The massager is water resistant and can be used in showers and bath tubs. Because of the relative simplicity of design and because of the use, to a large extent, of standardized parts, the system is relatively inexpensive to manufacture and to maintain. Moreover, the massager provides relatively deep penetration of invigorating massage. The massager provides a vibratory, muscular and skin massage for easing strain, tension, pain and the like and which includes replaceable head that moves with respect to the main housing of the massager during operation of the massager.

These and other advantages and features of the present invention will become more apparent from the following description of the preferred embodiment of the invention taken in conjunction with the accompanying drawings.

### BRIEF DESCRIPTION OF THE DRAWINGS

Referring particularly to the drawings for the purpose of illustration only and not limitation, there is illustrated:

FIG. 1 is an illustrative diagram showing a cleansing and massaging system;

FIG. 2 shows an exemplary component layout for the system of FIG. 1;

FIG. 3 shows a power sub-system for the system of FIG. 1;

FIG. 4 shows an exemplary assembled back cover, while an exemplary completed assembly of the system of FIG. 1 is shown in FIGS. 5-6;

FIG. 7 shows an exemplary cradle for charging the system of FIG. 1;

FIGS. 8-9 show an exemplary user interface for the system of FIG. 1;

FIG. 10 shows an exemplary circuit for the cleansing and massaging system of FIG. 1;

FIG. 11 is a side view of a handle body of a cleaning and massaging system;

FIG. 12 is a front view of the handle body of the cleaning and massaging system;

FIG. 13 is a back view of the handle body of the cleaning and massaging system;

FIG. 14 is a perspective view of the handle body of the cleaning and massaging system;

FIG. 15 is a perspective view of a cleaning head;

FIG. 16 is a side view of the handle body with the cleaning head;

FIG. 17 is a front view of the handle body with the cleaning head;

FIG. 18 is a perspective view of the handle body with the cleaning head;

FIG. 19 is a front view of the handle body with the cleaning head seated in a charging cradle;

FIG. 20 is a perspective view of a massaging head;

FIG. 21 is a side view of the handle body with the massaging head;

FIG. 22 is a front view of the handle body with the massaging head;

FIG. 23 is a perspective view of the handle body with the massaging head;

FIG. 24 shows an exemplary process to provide different massaging modes; and

FIGS. 25-26 show exemplary usage scenarios using the system described above.

### DESCRIPTION

Although specific embodiments of the present invention will now be described with reference to the drawings, it should be understood that such embodiments are by way of example only and merely illustrative of but a small number of the many possible specific embodiments which can represent applications of the principles of the present invention. Various changes and modifications obvious to one skilled in the art to which the present invention pertains are deemed to be within the spirit, scope and contemplation of the present invention as further defined in the appended claims.

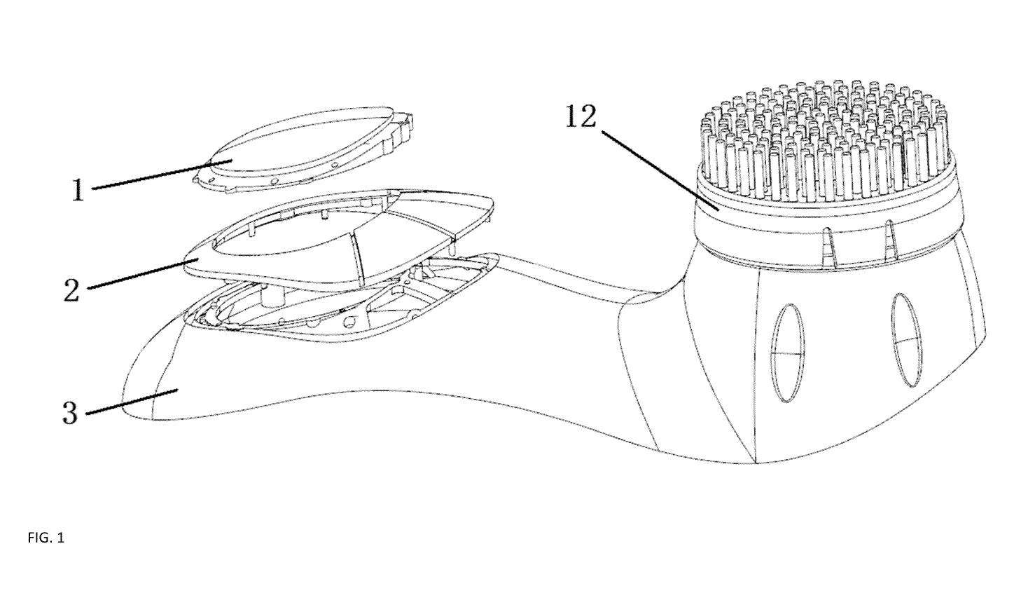

Referring now to FIGS. 1, 2 and 3, an embodiment of a cleansing and massaging system of the present invention is shown. The system includes a hollow molded plastic housing with upper and lower portions which are secured together by one or more screws. The housing includes an integral, lightly tapering handle portion which extends from a main, generally oval portion containing display and buttons into a wider oval head portion containing a user-replaceable head mount for brushes, among others.

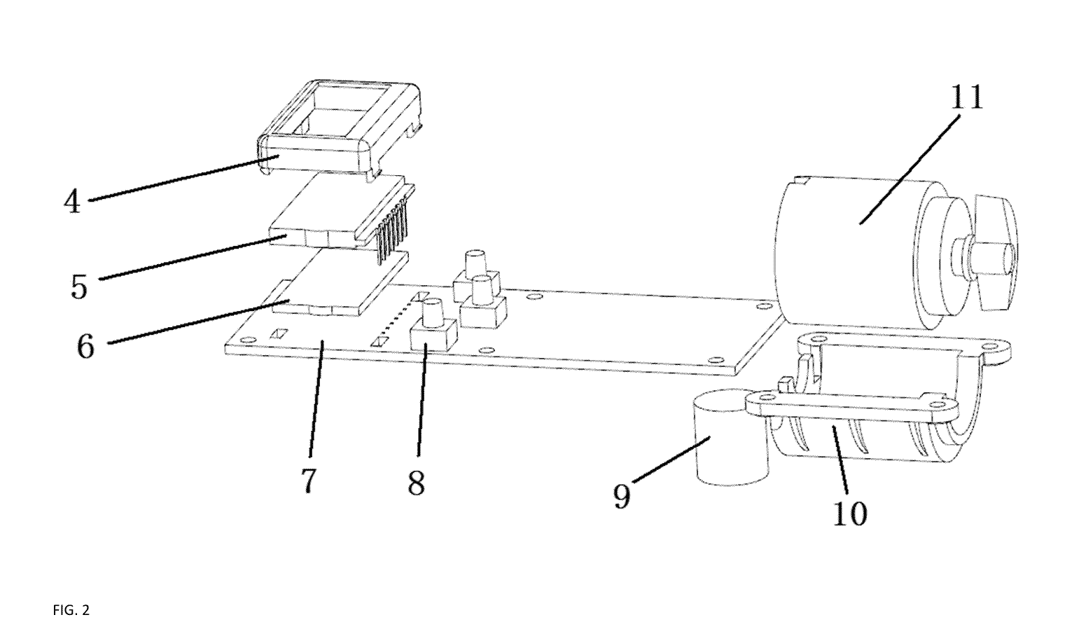

Referring now to FIG. 1, the cleansing and massaging system includes a front cover 3 that houses a display transparent cover 1 above a press button support 2. A body brush head 12 is connected to the front cover 3 to transmit vibratory motion generated by a vibration generator 11 (FIG. 2) to impinge on a user's face or body part. The vibration generator may include eccentric motors, solenoids, transducers, sonic transducers, vibratory means or any other suitable means known to one skilled in the art.

The system has with five different user-replaceable brush heads. Each brush is for a special skin type and body area:
Body Brush Head
Normal Skin Brush Head
Sensitive Skin Brush Head
Toning Applicator
Hydration/Exfoliation Applicator

The head is flexibly mounted on the main housing of the massager so that the body engaging surface of the head may follow body contours and so that vibratory motion may be imparted to the head. As a consequence, relatively deep penetration of invigorating, pulsating massage can be achieved for maximum comfort.

To take the brush head off device, the user can turn the brush counterclockwise and pull away. To attach the brush head on device, the user can slightly push down until a resistance is felt and the brush can then be locked in place.

The brush head should be cleaned once a week with antibacterial soap or liquid. To clean, remove the brush head from the device and wash with soapy, warm water. It is recommended to replace the brush head every 3-6 months (depending on how often you use it) as the brush bristles become worn and less effective over time.

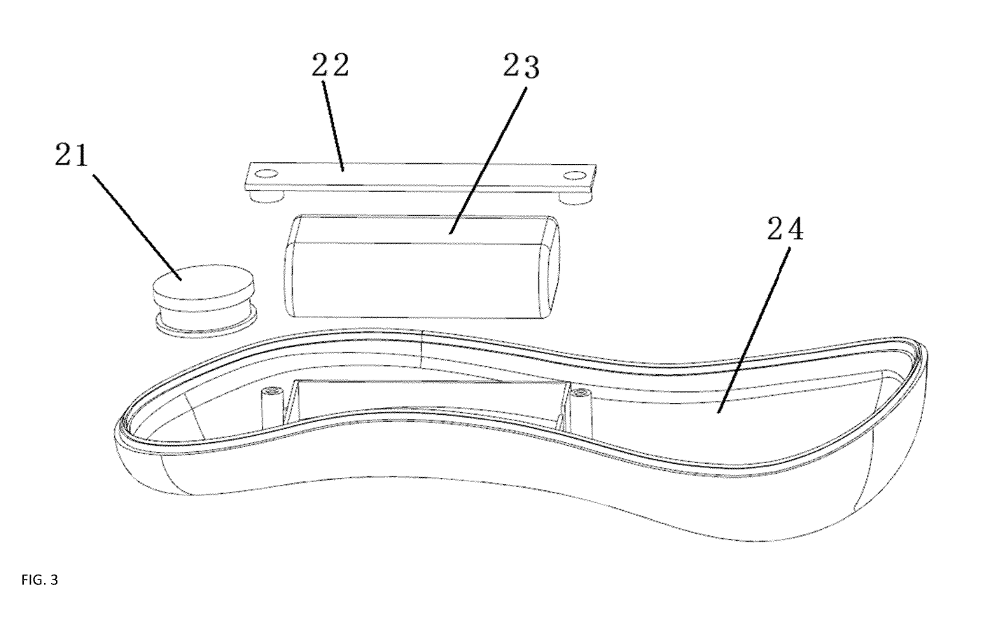

FIG. 2 shows an exemplary component layout for the system of FIG. 1. In this embodiment, a display support 4 is positioned above a display 5. The display 5 in turn is positioned above a backlit board 6 to light up the display 5 during use. The display 5 and backlit board 6 are soldered to a circuit board 7. One or more buttons 8 are positioned on the board to receive user inputs. The circuit board 7 is positioned above a back cover (FIG. 3) through a buffer 9. The circuit board 7 also controls a motor 11 for generating vibrations for the cleansing and massaging system. The motor 11 is supported by a motor support 10 and drives the brush 12 and transmits vibratory energy to the user. Various motor types can be used. For example, the motor could be replaced by a multi-speed motor which could then be used to impart different rates of vibratory motion to the head.

The motor 11 has an unbalanced head to form a vibrating motor, which is essentially a motor that is improperly balanced. In other words, there is an off-centered weight attached to the motor's rotational shaft that causes the motor to wobble. The amount of wobble can be changed by the amount of weight attached to the motor. The vibrating motor can be done by attaching any off centered weight to any motor shaft.

In one embodiment, the button is connected to a microcontroller, which in turn is connected to the motor 11 by means of wires and is mounted in the head portion so that it can be actuated by a person while he is holding the massager by the handle portion.

In one embodiment shown in FIG. 2, the vibration is generated by the motor driven blade. In another embodiment, the vibration can be generated by a motor-driven cam wheel which is coupled to a member to be vibrated. Another type of vibrator includes an eccentric weight rotationally driven by a motor which is fixed to a member to be vibrated. In this device, the member, together with the motor, is subjected to a revolving vibration (i.e., orbital movement) caused by centrifugal force generated by rotation of the eccentric weight.

It will be appreciated that the cushioning motor support shown in FIG. 2 is not limited to the configuration shown. It is emphasized that while the configuration shown in FIG. 2 is preferred, it is also within the spirit and scope of the present invention to have any different configurations not shown.

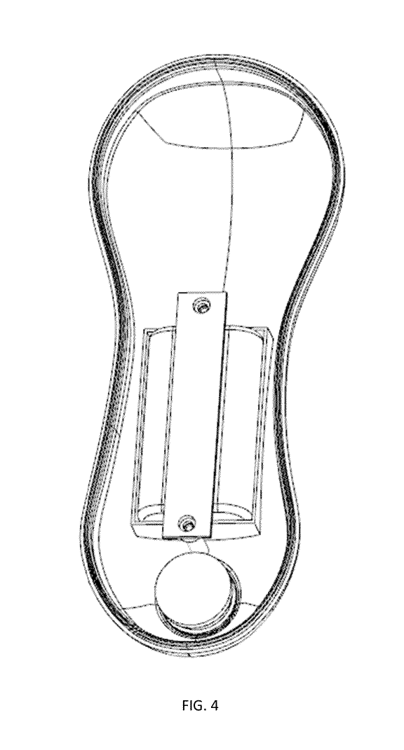

Turning now to FIG. 3, a power sub-system for the system of FIG. 1 is detailed. The power sub-system is mounted on a back cover 24 of the cleansing and massaging system. A rechargeable battery 23 is placed in a compartment on the back cover 24 and secured to the compartment through a fixture 22. The battery is recharged through an inductive coil 21. The assembled back cover 24 is shown in FIG. 4. The completed assembly is shown in FIGS. 5-6.

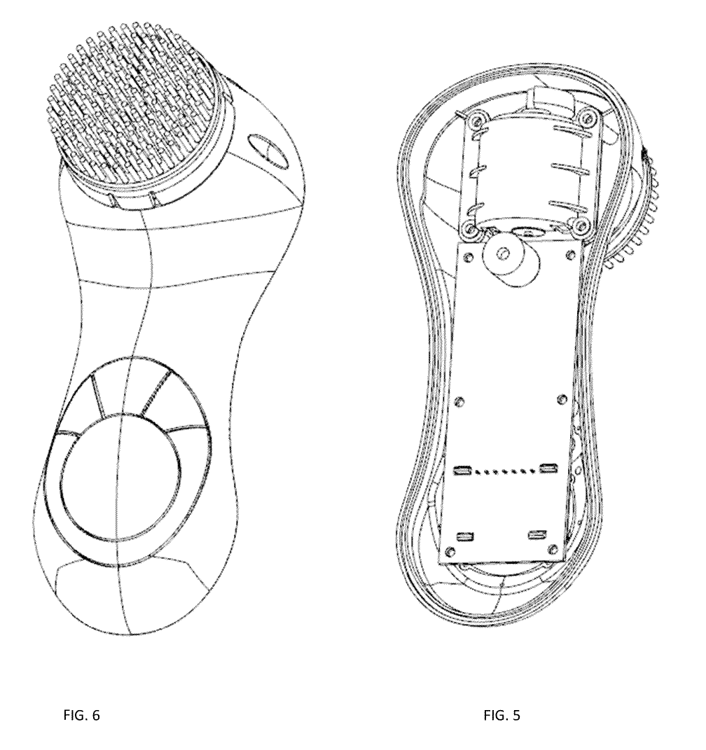

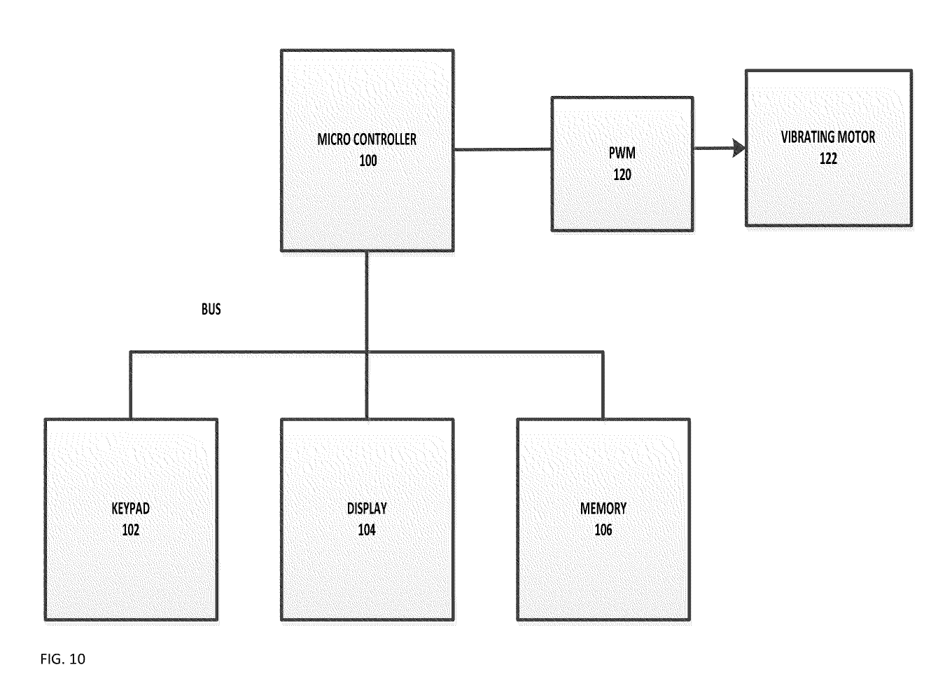

FIGS. 7-9 show an exemplary user interface for the system of FIG. 1. As shown in FIG. 9, the system has three buttons for the mode, on/off, and intensity, respectively. A battery charge level is also shown on the display. Once the system is charged, the system can be turned on or off by pushing the power button in the center of the button group. To turn the system off, the user can press the same button again. The mode selection is shown in the display of FIG. 10, along with the intensity level as a bar chart.

There are five levels strength adjustable. When the user presses the Intensity button, the default in intensity is intensity 1 for sensitive skin. The user can press the intensity button to change to intensity 2 for normal skin, to intensity 3 for deep pore skin, to intensity 4 & 5 for body skin. If the user keeps pressing the intensity button, the intensity level rotates from levels 1 to 5. If the user is unsure which brush head and intensity setting is best, testing each facial brush with different mode and intensity, the user can play with the setting on small area of his/her skin before using it over the face and neck. This will help determine the right combination for the skin and comfort level.

The mode button allows the user to selectably change the three types of cleansing therapeutic modes, as displayed on the clear and easy LCD screen.

FIG. 10 shows an exemplary circuit for the cleansing and massaging system of FIG. 1. A microcontroller 100 receives user input in the form of pushbuttons or keypad 102. The microcontroller 100 also provides visual feedback through a display 104. Instructions for the different massaging modes are stored in memory 106 which includes ROM and RAM. The microcontroller 100 also controls a motor 122 through a pulse width modulation circuit 120, although other motor control circuits can be used.

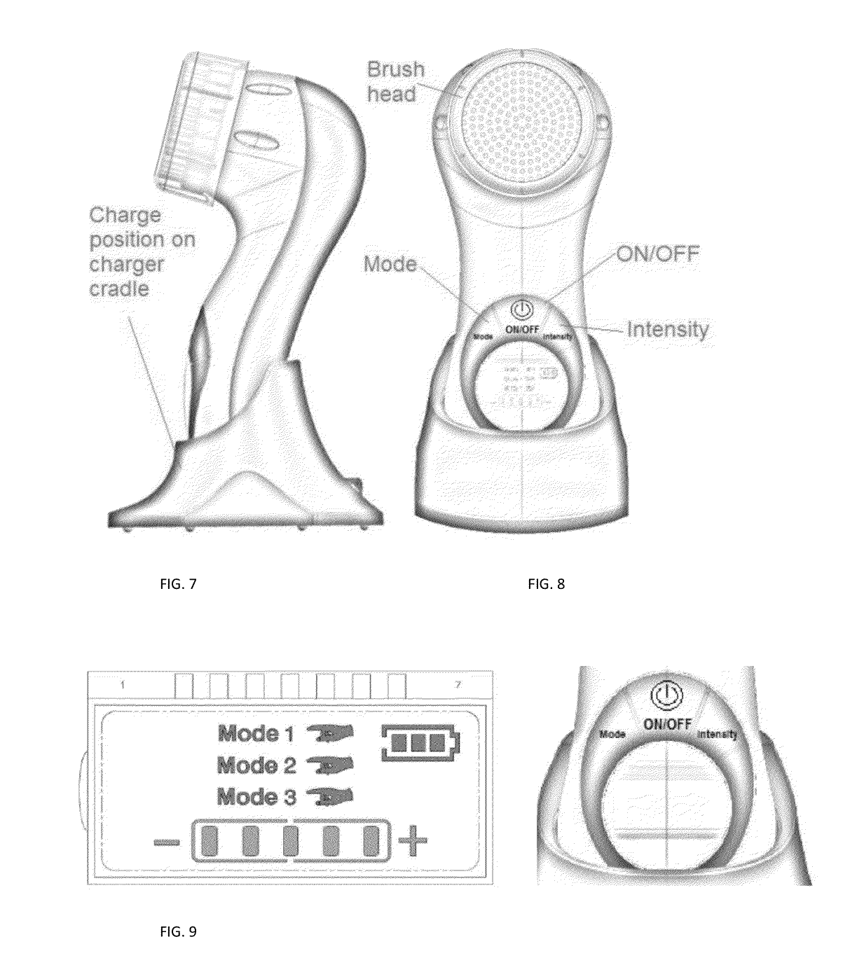

During operation, prior to first use, the system is charged through a cradle, as shown in FIG. 8. Preferably, the charging is done inductively, but the charging can also be done using a direct wired connection to an AC adapter.

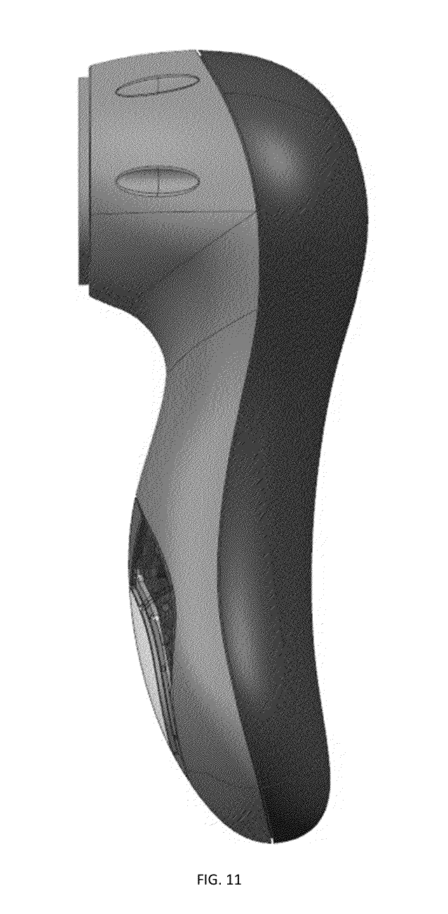

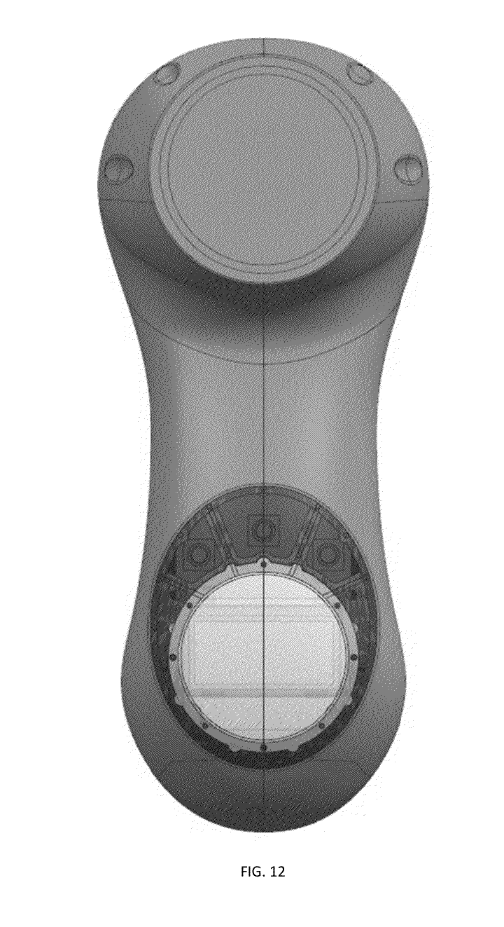

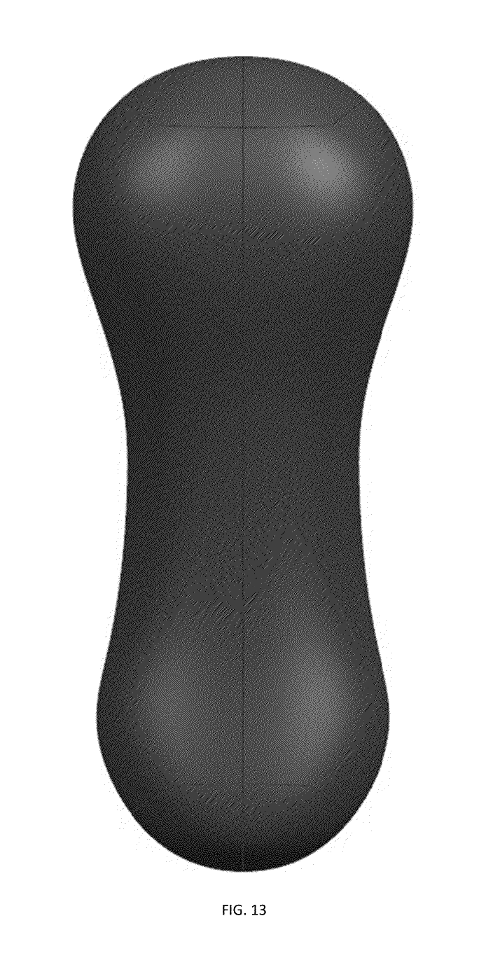

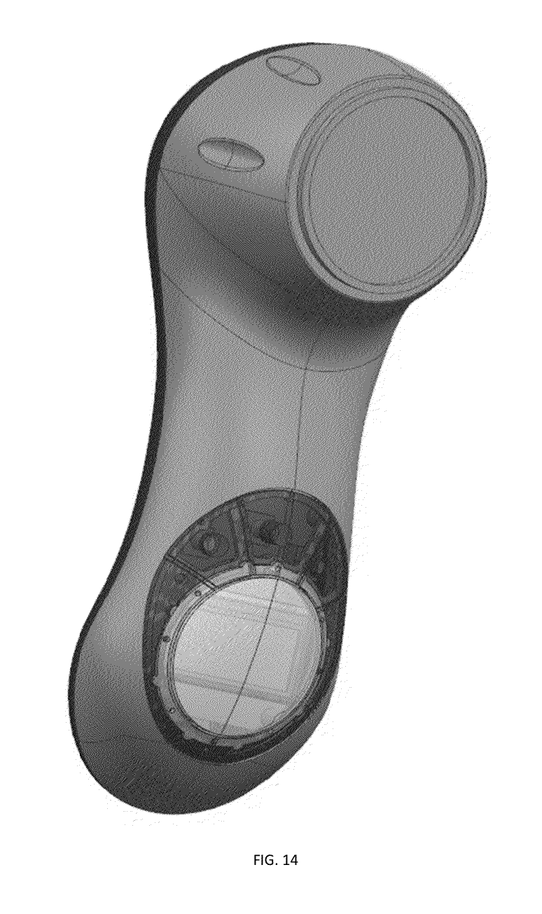

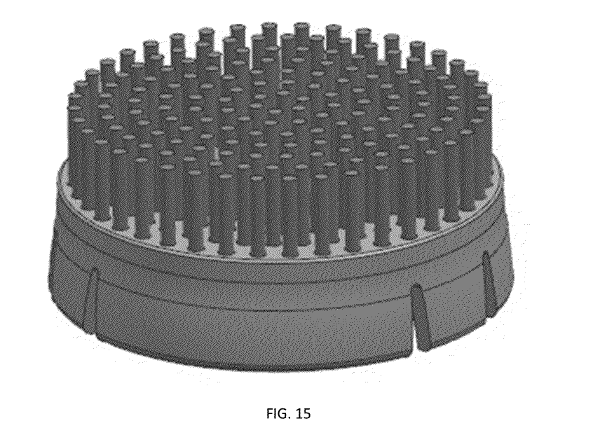

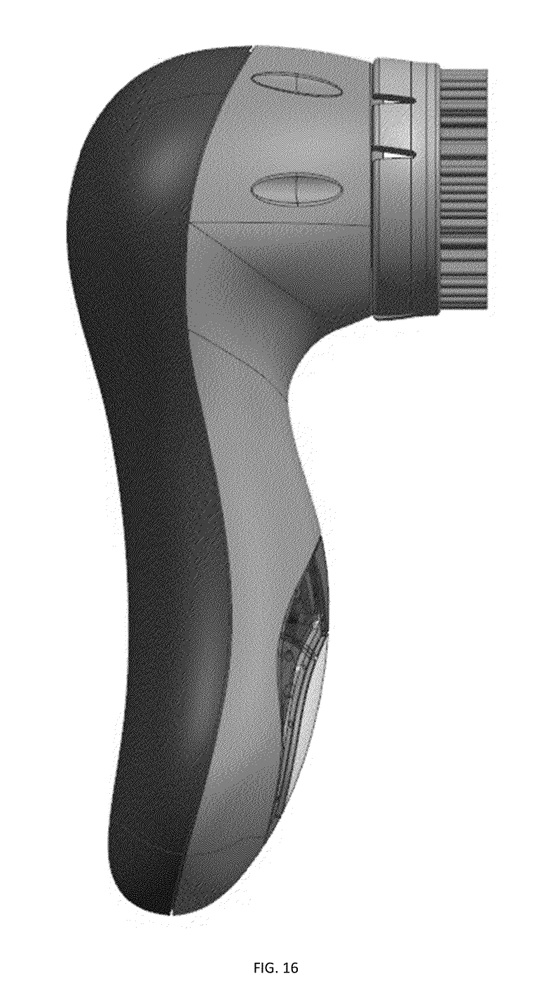

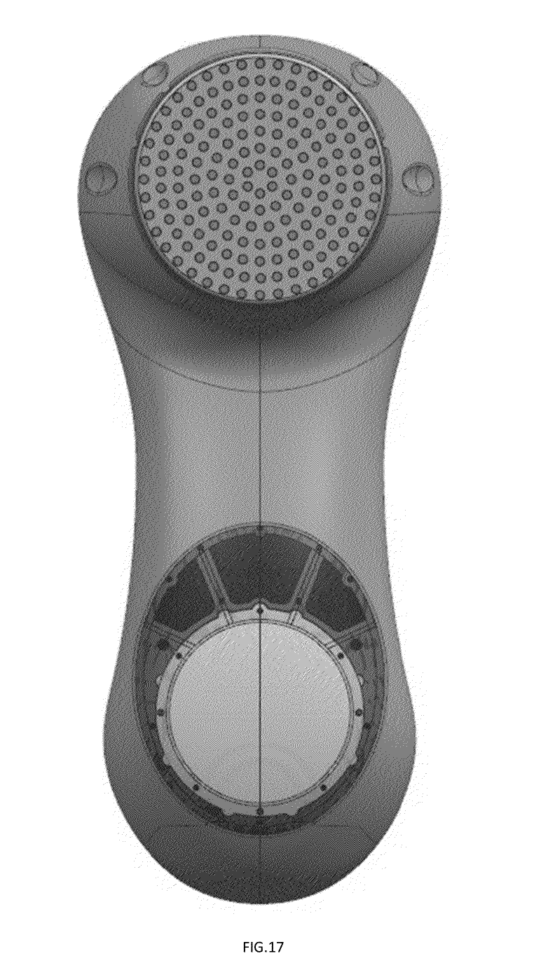

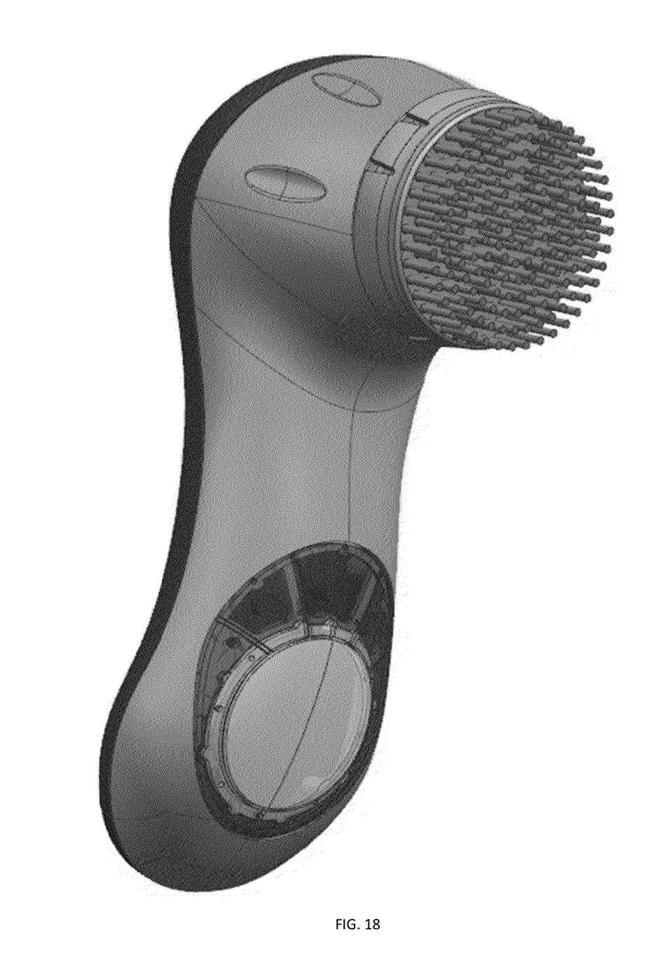

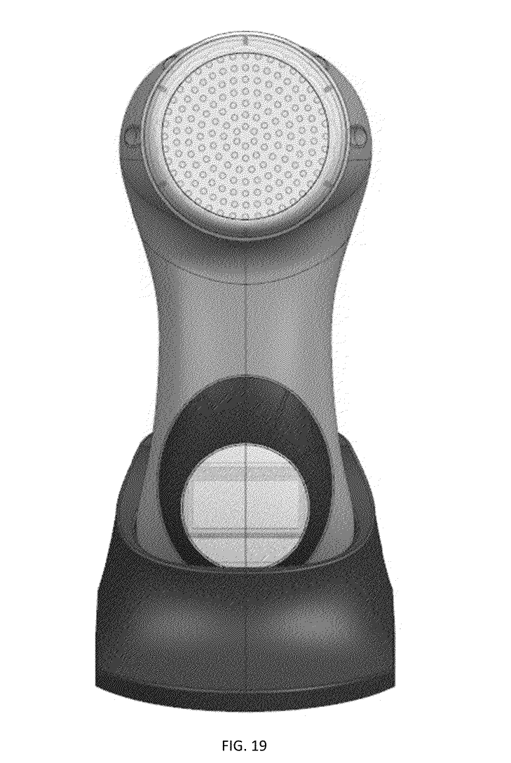

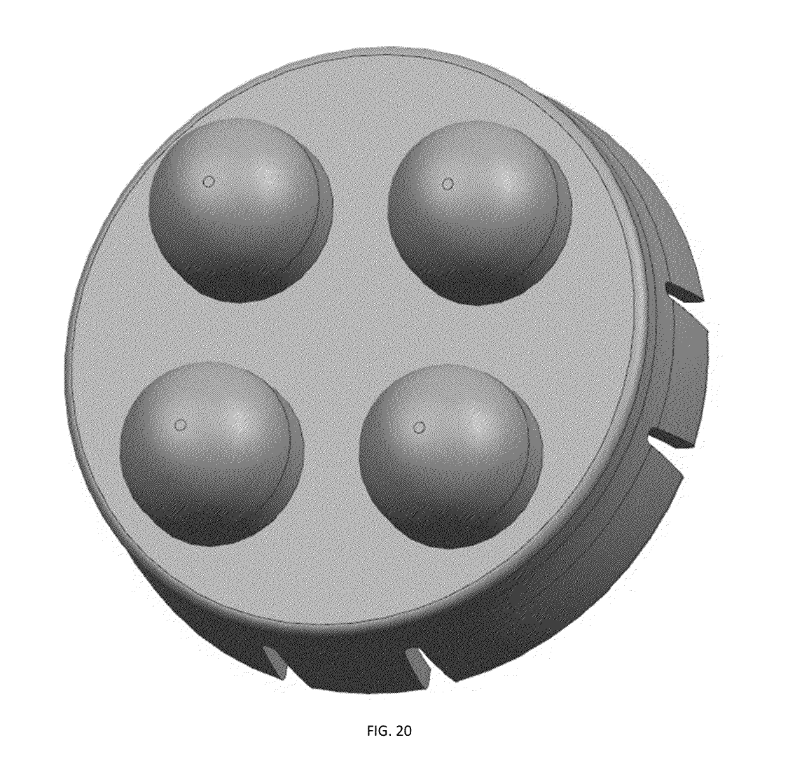

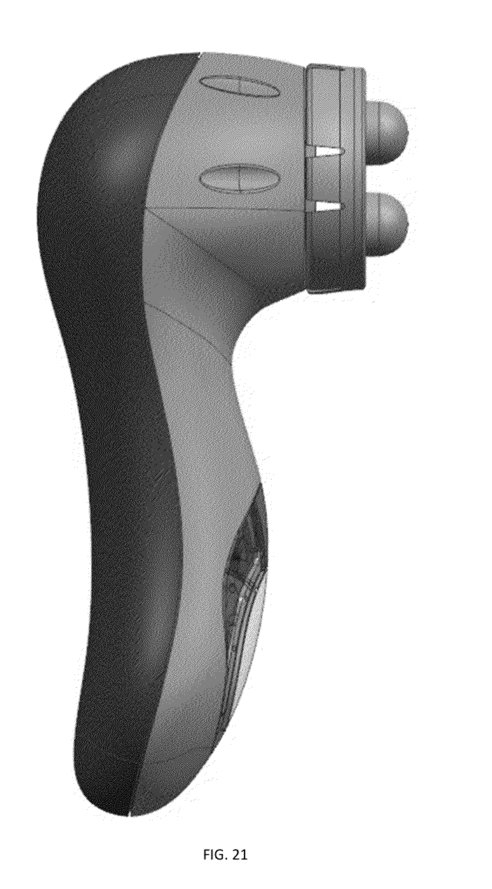

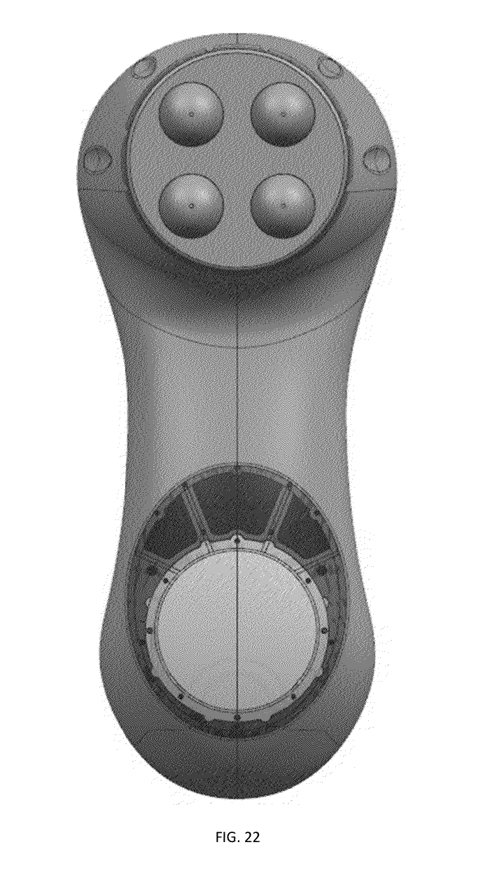

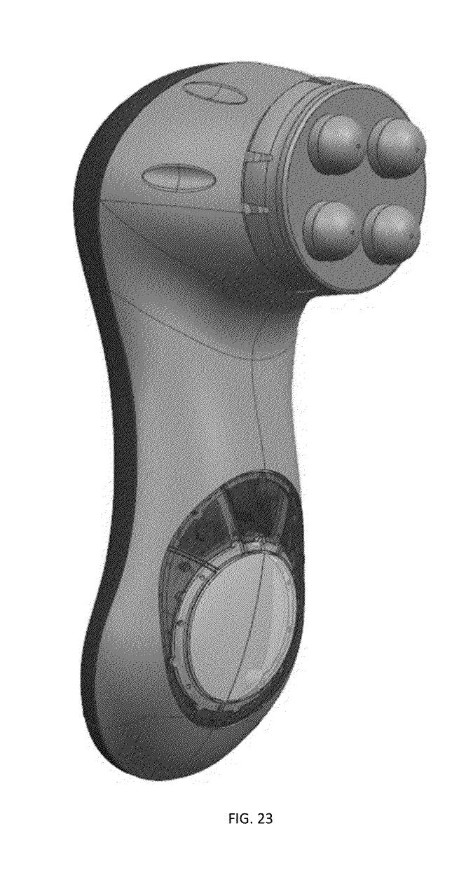

FIG. 11 is a side view of a handle body of a cleaning and massaging system. FIG. 12 is a front view of the handle body of the cleaning and massaging system. FIG. 13 is a back view of the handle body of the cleaning and massaging system. FIG. 14 is a perspective view of the handle body of the cleaning and massaging system. FIG. 15 is a perspective view of a cleaning head. FIG. 16 is a side view of the handle body with the cleaning head. FIG. 17 is a front view of the handle body with the cleaning head. FIG. 18 is a perspective view of the handle body with the cleaning head. FIG. 19 is a front view of the handle body with the cleaning head seated in a charging cradle. FIG. 20 is a perspective view of a massaging head. FIG. 21 is a side view of the handle body with the massaging head. FIG. 22 is a front view of the handle body with the massaging head. FIG. 23 is a perspective view of the handle body with the massaging head.

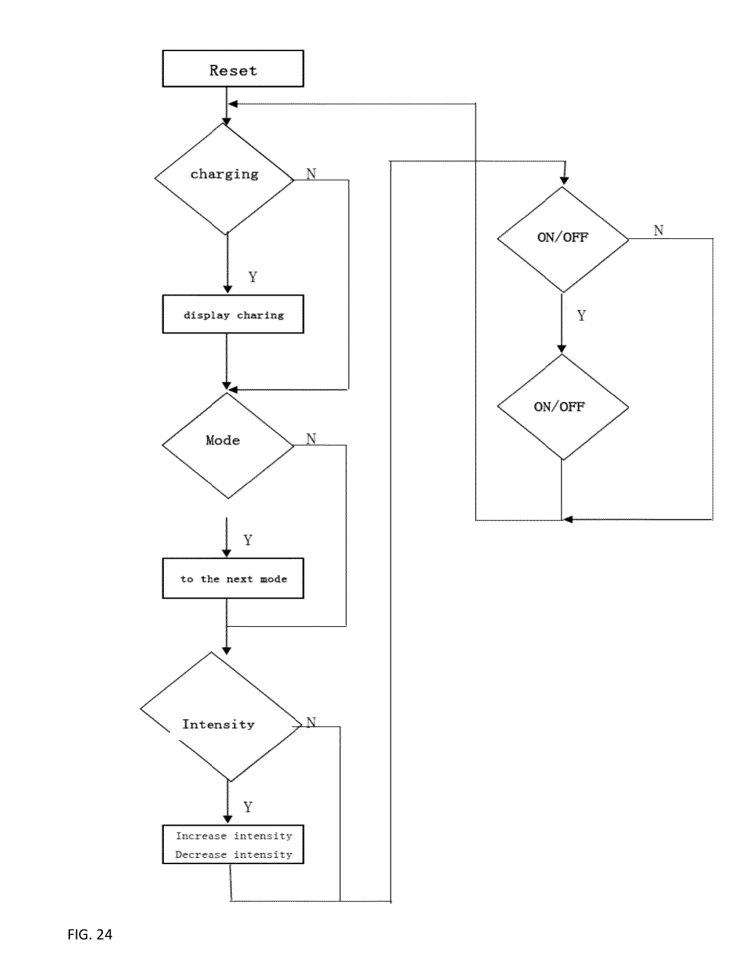

FIG. 24 shows an exemplary process to provide different massaging modes. They are:

1. Power on (Reset)

2. Detect if unit is charging. If it is charging, the battery capacity displays symbols on the screen, the process flashes the display bar alternately from left to right. When the battery is fully charged, (the three grids of the battery symbol are full), the process ceases to flash. If the state of charge is not detected, then the process enters directly into a standby mode.

3. In the wait mode, if the process detects that the user has pressed the MODE button, then the process enters the mode selection. One embodiment provides three modes:
a. Continuous mode: the motor has continuous action
b. Intermittent mode: motor work for 0.4 seconds, stop for 0.2 seconds
c. Mixed mode: the combination of the continuous mode, where the massage is performed for 10 seconds, stops for one second, and then performs the intermittent mode: discontinuous mode for 10 seconds, stop for 1 second.

4. Wait mode: if the process detects an intensity button actuation, then the process enters a motor intensity adjustment module. One embodiment offers 5 levels of intensity. The default mode is the lowest level. If the user continuously presses the intensity key, the process increases the intensity in ascending order of intensity to the fifth level intensity, and the levels continue to cycle upon button pressing. In the wait mode, the process detects the ON/OFF button actuation by the user and turns power on or off as specified.

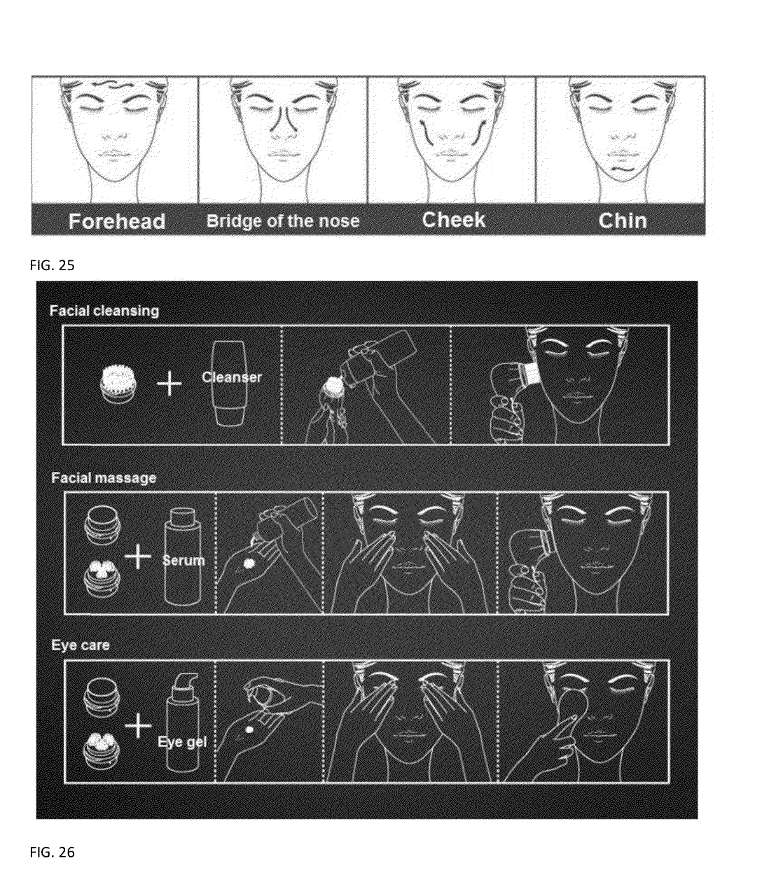

FIGS. 25-26 show exemplary usage scenarios using the system of FIG. 1. Make-ups should be removed prior to using the system as the make-up chemicals may stain the brush bristles. The user can wet the skin and the brush head with water or suitable fluid, and the user can apply cleansers directly to the skin or to the brush head. The brush is then gently moved over the face and neck area using small and slow circular motions. In one use case, the user begins with the forehead and work his/her way down the rest of the face and neck. The system is water proof and can be used in the shower or bathtub. However, the charger cradle should not be submerged in water during charging operation.

In view of the foregoing, it is apparent that the massager is a commercially attractive device from the standpoint that it provides a satisfying and comforting muscular and skin massage and that it is relatively inexpensive to manufacture and maintain.

Of course the present invention is not intended to be restricted to any particular form or arrangement, or any specific embodiment, or any specific use, disclosed herein, since the same may be modified in various particulars or relations without departing from the spirit or scope of the claimed invention hereinabove shown and described of which the apparatus or method shown is intended only for illustration and disclosure of an operative embodiment and not to show all of the various forms or modifications in which this invention might be embodied or operated.

The present invention has been described in considerable detail in order to comply with the patent laws by providing full public disclosure of at least one of its forms. However, such detailed description is not intended in any way to limit the broad features or principles of the present invention, or the scope of the patent to be granted. Therefore, the invention is to be limited only by the scope of the appended claims.

What is claimed is:

1. A cleaning and massaging system, comprising:
a water resistant housing having an elongated and curved top surface and a bottom surface, wherein the top surface has a water resistant display and input panel at one end and a user replaceable cleaning and massage head;
a display and one or more buttons mounted in the water resistant display and input panel, said display protected by a circular front cover with a transparent cover above a press button support;
a processor inside the housing;
a vibration generator driven by the processor to clean or massage on user command; and
computer readable code executed by the processor to receive at least three modes of operation and five modes of intensity and to control the vibration generator in accordance therewith.

2. The system of claim 1, wherein the vibration generator comprises a motor.

3. The system of claim 1, comprising an inductively charged battery to power the motor.

4. The system of claim 1, comprising one or more user-replaceable brush heads selected from a group consisting of: Body Brush Head, Normal Skin Brush Head, Sensitive Skin Brush Head, Toning Applicator, and Hydration or Exfoliation Applicator.

5. The system of claim 1, comprising a motor driven blade to generate vibration.

6. The system of claim 5, wherein the motor driven blade is eccentric.

7. The system of claim 1, wherein one of the buttons comprises an intensity button to select five different intensity levels.

8. The system of claim 7, wherein the intensity levels include intensity 1 for sensitive skin, intensity 2 for normal skin, intensity 3 for deep pore skin, and intensity 4 and 5 for body skin treatment.

9. The system of claim 1, wherein one of the buttons comprises a mode button to select three different modes.

10. The system of claim 1, wherein the user replaceable cleaning and massage head comprises a circular flat region coupled to a circular wall with four spaced apart V-shaped openings.

11. A processor controlled method for cleaning and massaging a person with a water resistant housing having a top surface and a bottom surface, wherein the top surface has a water resistant display and input panel at one end and a user replaceable cleaning and massage head, comprising:
providing a display and one or more buttons mounted in the water resistant display and input panel to receive at least three modes of operation and five modes of intensity and to control the vibration generator in accordance therewith; and
driving a vibration generator driven by the processor to clean or massage on user command.

12. The method of claim 11, comprising generating ultrasonic vibration with a motor.

13. The method of claim 11, comprising picking one or more user-replaceable brush heads selected from a group consisting of: Body Brush Head, Normal Skin Brush Head, Sensitive Skin Brush Head, Toning Applicator, and Hydration or Exfoliation Applicator.

14. The method of claim 11, comprising vibrating with a motor driven blade.

15. The method of claim 15, wherein the motor driven blade is eccentric.

16. The method of claim 11, wherein one of the buttons comprises an intensity button to select five different intensity levels.

17. The method of claim 17, wherein the intensity levels include intensity 1 for sensitive skin, intensity 2 for normal skin, intensity 3 for deep pore skin, and intensity 4 and 5 for body skin treatment.

18. The method of claim 11, wherein one of the buttons comprises a mode button to select three different modes.

19. The method of claim 11, comprising using the housing in a shower.

20. The method of claim 11, comprising inductively charging a battery powering the processor.
